(19) United States
(12) Patent Application Publication
(10) Pub. No.: US 2024/0238217 A1
(43) Pub. Date: Jul. 18, 2024

(54) USE OF CLOFOCTOL FOR THE TREATMENT OF INFLAMMATION

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); CENTRE HOSPITALIER REGIONAL UNIVERSITAIRE DE LILLE, Lille (FR); INSTITUT PASTEUR DE LILLE, Lille (FR); UNIVERSITÉ DE LILLE, Lille (FR); APTEEUS, Lille (FR)

(72) Inventors: François TROTTEIN, Lille Cedex (FR); Arnaud MACHELART, Lille (FR); Benoît DEPREZ, Lille (FR); Valentin SENCIO, Lille cedex (FR)

(21) Appl. No.: 18/559,411

(22) PCT Filed: May 6, 2022
(86) PCT No.: PCT/EP2022/062335

§ 371 Date: Nov. 7, 2023

(30) Foreign Application Priority Data
May 12, 2021 (EP) 21305619.5

(51) Int. Cl. A61K 31/05; A61P 29/00

(52) U.S. Cl. CPC A61K 31/05; A61P 29/00

(57) ABSTRACT

There is a need for novel therapies for the treatment of inflammation. Now, the inventors surprisingly show that clofoctol has potent anti-inflammatory properties. In particular, the inventors demonstrate that clofoctol treatment drastically reduced pulmonary inflammation in two models that recapitulate severe inflammation, one induced by the virus SARS-COV-2, and the other induced by LPS. Collectively, the data of the inventors justify to suggest that clofoctol would be suitable for the treatment of inflammation in general.

### FIELD OF THE INVENTION

The present invention is in the field of medicine, in particular inflammation.

### BACKGROUND OF THE INVENTION

The mucosa (including airway, intestinal, oral and cervical epithelium) is an integrated network of tissues, cells and effector molecules that protect the host from environmental insults and infections. Dysregulation of immunity at mucosal surfaces is thought to be responsible for the alarming global increase in inflammations such as those affecting the gastrointestinal system (Crohn's disease, ulcerative colitis and irritable bowel syndrome) and respiratory system (asthma, allergy and chronic obstructive pulmonary disorder). For instance, the respiratory epithelium is in permanent contact with the external environment, with a total exchange surface area of approximately 100 to 130 m². In non-pathological cases, it is continuously exposed through inhalation to various pathogens or particles that can induce epithelial lesions. Facing these lesions, the airway epithelium must be able to restore its integrity through repair and regeneration mechanisms in order to regain all its functions, in particular its defence and barrier functions. Accordingly in chronic inflammatory respiratory diseases such as cystic fibrosis, chronic obstructive pulmonary disease, asthma or allergies, cellular and functional balances may be disrupted, resulting in the formation of epithelial reshaping or remodelling areas with the presence of squamous metaplasia and/or basal or secretory cell hyperplasia. Accordingly, there is a need for novel therapies for the treatment of inflammation.

Clofoctol is a bacteriostatic antibiotic. It is used in the treatment of respiratory tract and ear, nose and throat infections caused by Gram-positive bacteria. The anti-inflammatory effect of clofoctol has never been reported.

### SUMMARY OF THE INVENTION

The present invention is defined by the claims. In particular, the present invention relates to use of clofoctol for the treatment of inflammation.

### DETAILED DESCRIPTION OF THE INVENTION

The inventors surprisingly show that clofoctol has anti-inflammatory properties.

Accordingly, the present invention relates to a method of treating inflammation in a subject in need thereof comprising administering to the subject a therapeutically effective amount of clofoctol.

In some embodiments, the present invention relates to a method of treating inflammation in a subject in need thereof comprising administering to the subject a therapeutically effective amount of clofoctol, wherein the inflammation is not secondary to a SARS-COV2 infection.

As used herein the terms “subject” or “patient” refers to a mammalian to which the present invention may be applied. Typically said mammal is a human, but may concern other mammals such as primates, dogs, cats, pigs, sheep, cows. In particular, the term “subject” refers to a mammalian patient, such as a human, who is confirmed to have an inflammation or who may be classified as having a probable or suspected case of having an inflammation. In some embodiments, the subject is a human infant. In some embodiments, the subject is a human child. In some embodiments, the subject is a human adult. In some embodiments, the subject is an elderly human. In some embodiments, the subject is a premature human infant.

As used herein, the term “inflammation” is used to describe the fundamental pathological process consisting of a dynamic complex of cytologic and histologic reactions that occur in tissues in response to an injury or abnormal stimulation caused by a physical, chemical or biologic agent (e.g. bacterium, virus . . . ) including the local reactions and resulting morphologic changes, the destruction or removal of the injurious material, and the responses that lead to repair and healing. The so-called cardinal signs of inflammation are redness, heat, swelling, pain and, in certain cases, inhibited or lost function of the target organ. The redness and warmth result from an increased amount of blood in the affected tissue, which is usually congested; swelling ordinary occurs from the congestion and exudation; pressure on (or stretching of) nerve endings as well as changes in osmotic pressure and pH which may lead to significant pain; the disturbance in function may result in impairment in movement or the actual destruction of an anatomic part or organ. In some embodiments, the inflammation is localized in the gastrointestinal tract, kidneys, liver, heart, skin, spleen, brain, kidney and/or pulmonary tract, especially the lungs is favorably treated by the method of the present invention. Especially inflammation localized to the oral mucosa e.g. buccal and sublingual; nasal mucosa; eye mucosa; genital mucosa; rectal mucosa; aural mucosa; lung mucosa; bronchial mucosa; gastric mucosa; intestinal mucosa; olfactory mucosa; uterine mucosa; and esophageal mucosa is favorably treated by the method of the present invention.

The method of the present invention is thus particularly suitable for the treatment of inflammatory diseases. As used herein, “inflammatory disease” means a clinical disorder in which inflammation is a prominent contributor to the clinical condition. Typically, the inflammatory disease is selected from the group consisting of asthma, chronic obstructive lung disease, pulmonary fibrosis, pneumonitis (including hypersensitivity pneumonitis and radiation pneumonitis), pneumonia, cystic fibrosis, psoriasis, arthritis/rheumatoid arthritis, rhinitis, pharyngitis, cystitis, prostatitis, dermatitis, allergy including hay fever, nephritis, conjunctivitis, encephalitis, meningitis, opthalmitis, uveitis, pleuritis, pericarditis, myocarditis, atherosclerosis, human immunodeficiency virus related inflammation, diabetes, osteoarthritis, psoriatic arthritis, inflammatory bowel disease (Crohn's disease, ulcerative colitis)/colitis, sepsis, vasculitis, bursitis, connective tissue disease, autoimmune diseases such as systemic lupus erythematosis (SLE), polymyalgia rheumatica, scleroderma, Wegener's granulomatosis, temporal arteritis, vasculitis, cryoglobulinemia, and multiple sclerosis, parasite-induced inflammation, fungi-induced inflammation, bacterial-induced inflammation, and viral induced inflammation.

In some embodiments, the inflammation affects the gastrointestinal system and typically includes inflammatory bowel diseases (IBD) such as Crohn's disease, ulcerative colitis and irritable bowel syndrome.

The term “inflammatory bowel disease” or “IBD” is used as a collective term for ulcerative colitis and Crohn's disease. The term “Crohn's disease” or “CD” is used herein to refer to a condition involving chronic inflammation of the gastrointestinal tract. Crohn's-related inflammation usually affects the intestines, but may occur anywhere from the mouth to the anus. CD differs from UC in that the inflammation extends through all layers of the intestinal wall and involves mesentery as well as lymph nodes. The disease is often discontinuous, i.e., severely diseased segments of bowel are separated from apparently disease-free areas. In CD, the bowel wall also thickens which can lead to obstructions and the development of fistulas and fissures are not uncommon. As used herein, CD may be one or more of several types of CD, including without limitation, ileocolitis (affects the ileum and the large intestine); ileitis (affects the ileum); gastroduodenal CD (inflammation in the stomach and the duodenum); jejunoileitis (spotty patches of inflammation in the jejunum); and Crohn's (granulomatous) colitis (only affects the large intestine). The term “ulcerative colitis” or “UC” is used herein to refer to a condition involving inflammation of the large intestine and rectum. In patients with UC, there is an inflammatory reaction primarily involving the colonic mucosa. The inflammation is typically uniform and continuous with no intervening areas of normal mucosa. Surface mucosal cells as well as crypt epithelium and submucosa are involved in an inflammatory reaction with neutrophil infiltration. Ultimately, this reaction typically progresses to epithelial damage and loss of epithelial cells resulting in multiple ulcerations, fibrosis, dysplasia and longitudinal retraction of the colon. In some embodiments, the method of the present invention is particularly suitable for the treatment of colonic Crohn's disease. As used herein, the term “colonic Crohn's disease”, alternatively referred to as colonic CD, as used herein, means Crohn's disease where the inflammation is substantially localized to the colon.

In some embodiments, the method of the present invention is particularly suitable for the treatment of skin inflammation. Examples of skin inflammatory diseases include, but are not limited to, acne, rosacea, folliculitis, perioral dermatitis, photodamage, skin aging, psoriasis, ichtiosis, chronic wounds, bed sores, keratosis piralis, scars, including surgical and acne scars, sebaceous cysts, inflammatory dermatoses, post inflammatory hyperpigmentation, xerosis, pruritis, lichen planus, nodular prurigo, eczema, and miliaria. Skin inflammatory diseases treatable in accordance with the invention also include, for example, chronic or acute skin inflammation, skin fibrosis, scleroderma, or a skin fibrotic disease or disorder. In some embodiments, the method herein disclosed is particularly suitable for the treatment of scleroderma, atopic dermatitis, nephrogenic fibrosing dermopathy, mixed connective tissue disease, scleromyxedema, scleredema, keloid, sclerodactyly, or eosinophilic fasciitis. In some embodiments, the method herein describes is particularly suitable for the treatment of photodermatitis. As used herein, the term “photodermatitis” has its general meaning in the art and refers to skin inflammation in response to UV radiation/light. This tissue response can include pain, irritation, itch, influx of inflammatory and pain-enhancing cells and tissue injury.

In some embodiments, the method of the present invention is particularly suitable for the treatment of kidney inflammation. In particular, the method of the present invention relates to the treatment of nephritis. As used herein, the term “nephritis” has its general meaning in the art and refers to the inflammation of the kidneys and may involve the glomeruli, tubules, or interstitial tissue surrounding the glomeruli and tubules More particularly, the method of the present invention is particularly suitable for the treatment of glomerulonephritis, membranoproliferative glomerulonephritis, interstitial nephritis, IgA nephropathy (Berger's disease), pyelonephritis, Lupus nephritis, Goodpasture's syndrome, Wegener's granulomatosis,

In some embodiments, the method of the present invention is particularly suitable for the treatment of lung inflammation.

As used herein, the expression “lung inflammation” refers to accumulation of inflammatory cells in airway tissue. As used herein, an agent is said to exert an anti-inflammatory effect if, when administered to the subject, the agent is capable of reducing said accumulation. Because the lung is a vital organ for gas exchange, excessive inflammation can be life threatening. This inflammation thus can render difficult for oxygen to pass through the alveoli into the bloodstream. Macroscopically, inflammation is characterized by redness, swelling, heat, pain, and loss of function. Microscopically, it is exhibited by vasodilation, increased vascular permeability, and inflammatory cell infiltration. Lung inflammation can be acute or chronic, and there are many possible causes, including exposures, infections, and diseases like asthma or bronchitis. Typically, acute lung inflammation is dominated by neutrophils, whereas chronic reactions involve mainly macrophages and lymphocytes.

In some embodiments, the subject suffers from cystic fibrosis. As used herein the term “cystic fibrosis” has its general meaning in the art and refers to an inherited autosomal disease associated with mutations to the gene encoding the cystic fibrosis transmembrane conductor regulator (CFTR). The method of the invention may be performed for any type of cystic fibrosis such as revised in the World Health Organisation Classification of cystic fibrosis and selected from the E84 group: mucoviscidosis, Cystic fibrosis with pulmonary manifestations, Cystic fibrosis with intestinal manifestations and Cystic fibrosis with other manifestations. In some embodiments, the subject harbours at least one mutation in the CFTR gene, including, but not limited to F508del-CFTR, R117H CFTR, and G551D CFTR (see, e.g., http://www.genet.sickkids.on.ca/cftr, for CFTR mutations).

In some embodiments, the subject suffers from asthma. As used herein, the term “asthma” refers to diseases that present as reversible airflow obstruction and/or bronchial hyper-responsiveness that may or may not be associated with underlying inflammation. Examples of asthma include allergic asthma, atopic asthma, corticosteroid naive asthma, chronic asthma, corticosteroid resistant asthma, corticosteroid refractory asthma, asthma due to smoking, asthma uncontrolled on corticosteroids and other asthmas as mentioned, e.g., in the Expert Panel Report 3: Guidelines for the Diagnosis and Management of Asthma, National Asthma Education and Prevention Program (2007) (“NAEPP Guidelines”), incorporated herein by reference in its entirety.

In some embodiments, the subjects suffer from COPD. As used herein, the term “COPD” refers to chronic obstructive pulmonary disease. The term “COPD” is generally applied to chronic respiratory disease processes characterized by the persistent obstruction of bronchial air flow. COPD patients can suffer from conditions such as bronchitis, cystic fibrosis, asthma or emphysema.

In some embodiments, the method of the present invention is particularly suitable for the treatment of myocarditis. As used herein, the term “myocarditis” has its general meaning in the art and refers to the inflammation of the heart muscle (myocardium). Myocarditis reduces cardiac ability to pump and thus cause rapid or abnormal heart rhythms (arrhythmias). As used, herein, the term “severe myocarditis” refers to myocarditis that requires intensive care treatment (e.g. treatment in an intensive care unit).

In some embodiments, the method of the present invention is particularly suitable for the treatment of inflammation secondary to infection caused by virus, bacteria, fungi and parasites.

In particular, the method of the present invention is particularly suitable for the treatment of inflammation secondary to viral infection. In some embodiments, the inflammation is caused by a virus selected from the group consisting of Arenaviruses (such as Guanarito virus, Lassa virus, Junin virus, Machupo virus and Sabia), Arteriviruses, Roniviruses, Astroviruses, Bunyaviruses (such as Crimean-Congo hemorrhagic fever virus and Hantavirus), Barnaviruses, Birnaviruses, Bornaviruses (such as Borna disease virus), Bromoviruses, Caliciviruses, Chrysoviruses, Coronaviruses (such as Coronavirus and SARS-Cov1 and SARS-Cov2), Cystoviruses, Closteroviruses, Comoviruses, Dicistroviruses, Flaviruses (such as Yellow fever virus, West Nile virus, Hepatitis C virus, and Dengue fever virus), Filoviruses (such as Ebola virus and Marburg virus), Flexiviruses, Hepeviruses (such as Hepatitis E virus), human adenoviruses (such as human adenovirus A-F), human astroviruses, human BK polyomaviruses, human bocaviruses, human coronavirus (such as a human coronavirus HKU1, NL63, and OC43), human enteroviruses (such as human enterovirus A-D), human erythrovirus V9, human foamy viruses, human herpesviruses (such as human herpesvirus 1 (herpes simplex virus type 1), human herpesvirus 2 (herpes simplex virus type 2), human herpesvirus 3 (Varicella zoster virus), human herpesvirus 4 type 1 (Epstein-Barr virus type 1), human herpesvirus 4 type 2 (Epstein-Barr virus type 2), human herpesvirus 5 strain AD169, human herpesvirus 5 strain Merlin Strain, human herpesvirus 6A, human herpesvirus 6B, human herpesvirus 7, human herpesvirus 8 type M, human herpesvirus 8 type P and Human Cyotmegalovirus), human immunodeficiency viruses (HIV) (such as HIV 1 and HIV 2), human metapneumoviruses, human papillomaviruses (such as human papillomavirus-1, human papillomavirus-18, human papillomavirus-2, human papillomavirus-54, human papillomavirus-61, human papillomavirus-cand90, human papillomavirus RTRX7, human papillomavirus type 10, human papillomavirus type 101, human papillomavirus type 103, human papillomavirus type 107, human papillomavirus type 16, human papillomavirus type 24, human papillomavirus type 26, human papillomavirus type 32, human papillomavirus type 34, human papillomavirus type 4, human papillomavirus type 41, human papillomavirus type 48, human papillomavirus type 49, human papillomavirus type 5, human papillomavirus type 50, human papillomavirus type 53, human papillomavirus type 60, human papillomavirus type 63, human papillomavirus type 6b, human papillomavirus type 7, human papillomavirus type 71, human papillomavirus type 9, human papillomavirus type 92, and human papillomavirus type 96), human parainfluenza viruses (such as human parainfluenza virus 1-3), human parechoviruses, human parvoviruses (such as human parvovirus 4 and human parvovirus B19), human respiratory syncytial viruses, human rhinoviruses (such as human rhinovirus A and human rhinovirus B), human spumaretroviruses, human T-lymphotropic viruses (such as human T-lymphotropic virus 1 and human T-lymphotropic virus 2), Human polyoma viruses, Hypoviruses, Leviviruses, Luteoviruses, Lymphocytic choriomeningitis viruses (LCM), Marnaviruses, Narnaviruses, Nidovirales, Nodaviruses, Orthomyxoviruses (such as Influenza viruses), Partitiviruses, Paramyxoviruses (such as Measles virus and Mumps virus), Picornaviruses (such as Poliovirus, the common cold virus, and Hepatitis A virus), Potyviruses, Poxviruses (such as Variola and Cowpox), Sequiviruses, Reoviruses (such as Rotavirus), Rhabdoviruses (such as Rabies virus), Rhabdoviruses (such as Vesicular stomatitis virus, Tetraviruses, Togaviruses (such as Rubella virus and Ross River virus), Tombusviruses, Totiviruses, Tymoviruses, and Noroviruses among others. In some embodiments, the inflammation is not caused by a SARS-COV2 infection.

In some embodiments, the method of the present invention is particularly suitable for the treatment of a gastrointestinal inflammatory disease caused by a viral, bacterial or parasitic infection. For instance, bacterial infections include infections by Mycobacterium avium subspecies paratuberculosis, Clostridium difficile, Escherichia coli, Listeria monocytogenes, Campylobacter concisus. Examples of viruses that cause gastrointestinal infection include those from the families Toroviridae, Picobirnaviridae, Reoviridae, Adenoviridae, Coronaviridae or Picornaviridae, for example. In some embodiments, the viruses are astrovirus, Breda virus, human rotavirus, enteric adenovirus, human enteric coronavirus, or human enterovirus. In some embodiments, the gastrointestinal infection is caused by the SARS-COV2. In some embodiments, the gastrointestinal infection is not caused by the SARS-COV2.

In some embodiments, the method of the present invention is particularly suitable for the treatment of cholangitis. As used herein, the term “cholangitis” has its general meaning in the art and refers to an inflammation of the bile duct system. The bile duct system carries bile from your liver and gallbladder into the first part of your small intestine (the duodenum). In most cases cholangitis is caused by a bacterial infection, such as infections caused by Clostridium and Bacteroides, but it can have autoimmune or metabolic causes.

In some embodiments, the method of the present invention is particularly suitable for the treatment of sepsis. As used herein, the term “sepsis” has its general meaning in the art and represents a serious medical condition that is characterized by a whole-body inflammatory state. In addition to symptoms related to the provoking infection, sepsis is characterized by presence of acute inflammation present throughout the entire body, and is, therefore, frequently associated with fever and elevated white blood cell count (leukocytosis) or low white blood cell count and lower-than-average temperature, and vomiting. In particular, sepsis is defined as a deregulated immune response to infection, translating into life-threatening organs dysfunction, defined by a Sequential Organ Failure Assessment score of 2 more. Infection can be suspected or proven, or a clinical syndrome pathognomonic for infection. Septic shock is defined by infection and the need for vasopressors to maintain mean blood pressure≥65 mmHg and arterial lactate levels>2 mmol/l.

In some embodiments, the method of the present invention is particularly suitable for the treatment of lung inflammation that results from a lung infection. As used herein, the term “lung infection” has its general meaning in the art and means the invasion of lung tissues of a patient by disease-causing microorganisms, their multiplication and the reaction of lung tissues to these microorganisms and the toxins that they produce. In some embodiments, the patient suffers from a chronic lung infection. As used herein, the term “chronic infection” refers to a long-term infection which may be an apparent, unapparent or latent infection. In some embodiments, the patient suffers from an acute lung infection. As used herein, the term “acute lung infection” has its general meaning in the art and refers to a disease of the lungs characterized by inflammation and consolidation followed by resolution and caused by infection from viruses, fungi, or bacteria. The term is also known as “pneumonia”. Typically acute lung infection is associated with lung inflammation that is the rapid onset of progressive malfunction of the lungs, and is usually associated with the malfunction of other organs due to the inability to take up oxygen. In some embodiments, the lung infection is a bacterial infection, such as bacterial pneumonia. In some embodiments, the bacterial infection is caused by a bacterium selected from the group consisting of Streptococcus pneumoniae (also referred to as pneumococcus), Staphylococcus aureus, Streptococcus agalactiae, Streptococcus pyogenes, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Burkholderia cepacia, Burkholderia pseudomallei, Bacillus anthracis, Bacillus cereus, Bordetella pertussis, Stenotrophomonas maltophilia, a bacterium from the citrobacter family, a bacterium from the ecinetobacter family, and Mycobacterium tuberculosis or Mycobacterium abscessus. In some embodiments, the lung infection is a fungal infection. In some embodiments, the fungal infection is caused by a fungus selected from the group consisting of Histoplasma capsulatum, Cryptococcus neoformans, Pneumocystis jiroveci, Coccidioides immitis, Candida albicans, and Pneumocystis jirovecii (which causes pneumocystis pneumonia (PCP), also called pneumocystosis). In some embodiments, the lung infection is a viral infection, such viral pneumonia. In some embodiments, the viral infection is caused by a virus selected from the group consisting of influenza virus (e.g., Influenza virus A, Influenza virus B), respiratory syncytial virus, adenovirus, metapneumovirus, cytomegalovirus, parainfluenza virus (e.g., hPIV-1, hPIV-2, hPIV-3, hPIV-4), rhinovirus, coxsackie virus, echo virus, herpes simplex virus, coronavirus (SARS-coronavirus such as SARS-Cov1 or SARS-Cov2), and smallpox. In some embodiments, the lung infection is not caused by SARS-COV2. In some embodiments, the viral lung infection may be due to a member of the Pneumoviridae, Paramyxoviridae and/or Coronaviridae families and are in particular selected from the group consisting of upper and lower respiratory tract infections due to: human respiratory syncytial virus (hRSV), type A and type B, human metapneumovirus (hMPV) type A and type B; parainfluenza virus type 3 (PIV-3), measles virus, endemic human coronaviruses (HCoV-229E, -NL63, -OC43, and -HKU1), severe acute respiratory syndrome (SARS) and Middle-East respiratory syndrome (MERS) coronaviruses. In some embodiments, the coronavirus is not SARS-COV2. In particular, the method of the present invention is suitable for the treatment of Severe Acute Respiratory Syndrome (SARS). More particularly, the method of the present invention is suitable for the treatment of lung inflammation in patients suffering from COVID-19. In some embodiments, the patient does not suffer from COVID-19.

In some embodiments, the method of the present invention is particularly suitable for the treatment of an acute respiratory distress syndrome. The term “acute respiratory distress syndrome” (abbreviated ARDS), as used herein, relates to a severe, life-threatening medical condition characterized by presence of a risk factor (e.g. pneumoniapancreatitis, etc.), bilateral pulmonary infiltrates, and oxygen impairment not fully explained by cardiac failure. More specifically, the term ARDS as used herein relates to acute respiratory distress syndrome as convened in 2011 in the Berlin definition (ARDS Definition Task Force et al. 2012 JAMA 307(23): 2526-2533).

In some embodiments, the method of the present invention is particularly suitable for the treatment of the multisystem inflammatory syndrome.

As used herein, the term “multisystem inflammatory syndrome” or “MIS-C” has its general meaning in the art and refers to the inflammatory syndrome described in Whittaker, E., Bamford, A., Kenny, J., Kaforou, M., Jones, C. E., Shah, P., Ramnarayan, P., Fraisse, A., Miller, O., Davies, P., et al. (2020). Clinical Characteristics of 58 Children With a Pediatric Inflammatory Multisystem Syndrome Temporally Associated With SARS-COV-2. JAMA 324, 259-269. The term is also known as “Pediatric Inflammatory Multisystem Syndrome Temporally Associated with SARS-COV-2” or “PIMS-TS”. MIS-C is a condition where different body parts can become inflamed, including the heart, lungs, kidneys, brain, skin, eyes, or gastrointestinal organs. Subjects with MIS-C may have a fever and various symptoms, including abdominal (gut) pain, vomiting, diarrhea, neck pain, rash, bloodshot eyes, or feeling extra tired.

More particularly, the method of the present invention is particularly suitable for the treatment of a multisystem inflammatory syndrome (MIS-C) with severe myocarditis.

In some embodiments, the method of the present invention is particularly suitable for the prevention of inflammation-induced fibrosis.

As used herein, the expression “inflammation-induced fibrosis” relates to fibrosis developing during inflammatory diseases i.e. diseases related to acute or chronic inflammation (caused by tissue injury, pathogen infections or toxic agents) or as a consequence.

As used herein, the term “clofoctol” has its general meaning in the art and refers to the molecule having the IUPAC name 2-[(2,4-dichlorophenyl)methyl]-4-(2,4,4-trimethylpentan-2-yl)phenol (CAS Number 37693-01-9; PubChem CID 2799).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

By a “therapeutically effective amount” is meant a sufficient amount of clofoctol for the treatment of the lung inflammation at a reasonable benefit/risk ratio applicable to any medical treatment. It will be understood that the total daily usage of the compound will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 1 to 3,000 mg per adult per day.

In some embodiments, clofoctol is administered in combination with a corticosteroid. As used, the term “corticosteroid” has its general meaning in the art and refers to class of active ingredients having a hydrogenated cyclopentoperhydrophenanthrene ring system endowed with an anti-inflammatory activity. Corticosteroid drugs typically include cortisone, cortisol, hydrocortisone (11β, 17-dihydroxy, 21-(phosphonooxy)-pregn-4-ene, 3,20-dione disodium), dihydroxycortisone, dexamethasone (21-(acetyloxy)-9-fluoro-1β, 17-dihydroxy-16α-m-ethylpregna-1,4-diene-3,20-dione), and highly derivatized steroid drugs such as beconase (beclomethasone dipropionate, which is 9-chloro-11-β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone. corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

Typically the active ingredient of the present invention (i.e. the Clofoctol) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. In some embodiments, the pharmaceutical composition of the invention is administered topically (i.e. in the respiratory tract of the subject). Therefore, the compositions can be formulated in the form of a spray, aerosol, solution, emulsion, or other form well-known to one of skill in the art. If the method of the invention comprises intranasal administration of a composition, the composition can be formulated in an aerosol form, spray, mist or in the form of drops. In particular, the active ingredients for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

The invention will be further illustrated by the following figures and examples. However, these examples and figures should not be interpreted in any way as limiting the scope of the present invention.

### FIGURES

FIG. 1: Pharmacokinetics and inflammatory properties of clofoctol in a mouse model of COVID-19. a) Pharmacokinetics characterization of clofoctol in mice. Left panel, Female 8-10 week-old C57BL/6J mice were treated i.p. with a single dose of clofoctol (62.5 mg/kg) and were sacrificed at different time points thereafter. Right panel, Clofoctol was inoculated twice daily during two days and mice were sacrificed 1 h after the last injection. Clofoctol concentrations in lungs (n=3/time point, 3 samples/lung) and plasma (n=2/time point, technical replicates) are depicted. b-d) Effects of clofoctol treatment on SARS-COV-2 infection in K18-hACE2 transgenic C57BL/6J mice. b) Left panel, Scheme of the experimental design in which the effects of clofoctol was assessed in mice. Female K18-hACE2 transgenic mice were treated i.p. with clofoctol (62.5 mg/kg) or vehicle 1 h and 8 h after i.n. inoculation of SARS-COV-2 (5×10² TCID₅₀ per mouse) and twice daily at day 1 post-infection. Animals were sacrificed at day 2 and day 4 post-infection. The viral load was determined by titration on Vero-81 cells (middle panel) and by RT-qPCR (right panel) (day 2 post-infection). c) mRNA copy numbers of genes were quantified by RT-qPCR. Data are expressed as fold change over average gene expression in mock-infected animals (day 2 post-infection). d) Lung sections were analyzed at day 4 post-infection. Blinded sections were scored for levels of pathological severity. The inflammatory score is depicted. b-c) Results are expressed as the mean±SD (n=13 for panels b and c and n=6-7 for panel d). Significant differences were determined using the Mann-Whitney U test (**p<0.01; ***p<0.001).

FIG. 2: Effects of clofoctol treatment in female and male K18-hACE2 transgenic mice. a and b) The same operation was repeated but this time in male K18-hACE2 transgenic mice (clofoctol was injected at 50 mg/kg). Mice were sacrificed at day 2 post-infection. a) The infectious viral load (left panel) and viral RNA yields (right panel) in lungs are depicted. b) mRNA copy numbers of genes were quantified by RT-qPCR. c-e) Female mice were treated and infected as described in EXAMPLE. Mice were sacrificed at day 4 post-infection. c) the viral load was determined by RT-qPCR. d and e) mRNA copy numbers of genes were quantified by RT-qPCR (lungs). Data are expressed as fold change over average gene expression in mock-infected animals. Results are expressed as the mean±SD (n=6-7). Results are expressed as the mean±SD (n=5-7). Significant differences were determined using the Mann-Whitney U test (*p<0.05; **p<0.01).

FIG. 3: Effects of clofoctol treatment on LPS-induced cytokine production by alveolar macrophages. ELISA of IL-1β, IL-12p40, TNFα, and MCP-1 from supernatant of MPI cells pre-treated or not with clofoctol and stimulated or not with LPS during 24 h. The percentage cell viability post treatment is indicated. Significant differences were determined using the two-way ANOVA corrected for Dunnett's multiple comparisons test (viability test) or one-way ANOVA corrected for Dunnett's multiple comparisons test (n=7-11) (**p<0.01; ***p<0.001, ****p<0.0001).

FIG. 4: Effects of clofoctol treatment on LPS-induced cytokine mRNA expression by PMA-differentiated THP-1 macrophages. a, b, c and d) mRNA expression of MCP1, TNFα, IL-6 and IL-1β were quantified by RT-qPCR. Data are expressed as a fold-increase over the mean gene expression level in solvent control wells. (mean+/−SD from 3 experiments in duplicate). Significant differences were determined using the one-way ANOVA corrected for Dunnett's multiple comparisons test (**p<0.01; ***p<0.001, ****p<0.0001). ELISA of MCP1, TNFα and IL-6 (e, f and g) from supernatant of THP-1 cells pre-treated or not with Clofoctol or Dexamethasone and stimulated with LPS during 24 h. Results are expressed as the concentration of cytokine in pg/mL. (mean from one experiment in duplicate).

FIG. 5: Effects of clofoctol treatment on LPS-induced lung inflammation. Male C57BL/6J mice were treated i.p. with clofoctol (50 mg/kg) or vehicle 1 h before and 8 h after i.n. inoculation of LPS (10 μg/mouse) or PBS. Animals were sacrificed 24 h after LPS inoculation. mRNA copy numbers of genes were quantified by RT-qPCR. Data are expressed as fold change over average gene expression in PBS-treated animals. Results are expressed as the mean±SD (n=6). Significant differences were determined using the Mann-Whitney U test (**p<0.01).

FIG. 6: Effects of clofoctol treatment on IAV infection in mice. a) Upper panel, Scheme of the experimental design in which the effects of clofoctol was assessed in mice. Mice were treated i.p. with clofoctol (50 mg/kg) or vehicle 1 h and 8 h after i.n. inoculation of IAV and treated again twice at day 1 post-infection. Animals were sacrificed at day 2 and day 4 post-infection. Lower panel, The viral load was determined by titration on MDCK cells (left panel) and by RT-qPCR (right panel) (day 2 and 4 post-infection). b) Upper panel, mRNA copy numbers of genes were quantified by RT-qPCR. Data are expressed as fold change over average gene expression in mock-treated (uninfected) animals (day 2 post-infection). Lower panel, Cytokine concentration was measured by ELISA (4 dpi). c) Upper panel, Lung sections were analyzed at day 4 post-infection. Shown are representative lungs (hematoxylin and eosin staining). Lower pane, Blinded sections were scored for levels of pathological severity. The inflammatory score is depicted. Results are expressed as the mean±SD (n=6-8). Significant differences were determined using the Mann-Whitney U test (**p<0.01; ***p<0.001).

FIG. 7: Effects of clofoctol treatment on long-term sequela (fibrosis) imposed by IAV infection. a) Upper panel, Scheme of the experimental design. Mice were treated i.p. with clofoctol (50 mg/kg) or vehicle 1 h after i.n. inoculation of IAV and treated again at day 1, 2 and 3 post-infection. Animals were sacrificed at day 28. Lower panel, Body weight curves are shown. b) Lung sections were analyzed at day 28 post-infection (Red Sirius staining). Blinded sections were scored for levels of pathological severity. Results are expressed as the mean±SD (n=6-8).

### EXAMPLE 1

### Clofoctol Lowers Inflammation Induced by the SARS-COV-2

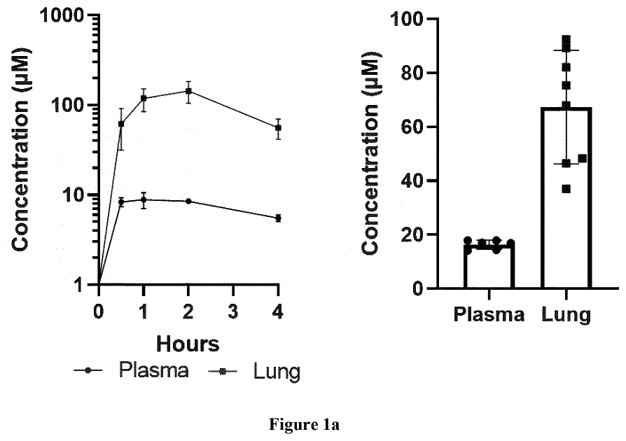

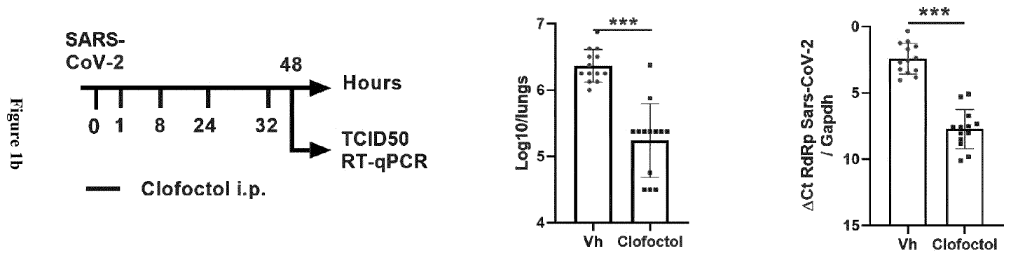

We use transgenic mice expressing the human angiotensin II-converting enzyme 2 (ACE2) receptor driven by the cytokeratin-18 (K18) gene promoter (K18-hACE2) as a model of SARS-CoV-2 infection wherein a decline in pulmonary function occurs and correlates with infiltration of monocytes, neutrophils and activated T cells. This model recapitulates the severe inflammation induced by the virus (Winkler, E. S., Bailey, A. L., Kafai, N. M. et al. SARS-CoV-2 infection of human ACE2-transgenic mice causes severe lung inflammation and impaired function. Nat Immunol 21, 1327-1335 (2020)). Before testing the potential antiviral activity of clofoctol, pharmacokinetic experiments were performed in female C57BL/6 mice. Clofoctol was injected intraperitoneally (i.p.) at 62.5 mg/kg to reach a lung concentration close to that achieved in humans at approved posology. Mice were sacrificed at 30 min, 1 h, 2 h and 4 h after i.p. administration of clofoctol. As early as 30 min after injection, clofoctol reached concentrations up to 61 μM in the lungs and remained above this level for almost 4 h (FIG. 1a, left panel), whereas it remained at a concentration seven times lower in the plasma. According to its expected half-life in the lungs, clofoctol concentration was anticipated to remain above its in vitro measured IC₉₅ (IC₉₅˜10 μM) for more than 7 consecutive hours. It was then decided to treat the mice twice daily to maintain a lung concentration close to 60 μM. In this setting, clofoctol concentration reached 67 μM in the lungs, 1 h after the fourth administration (FIG. 1a, right panel). Because of this favorable pharmacokinetic profile in mice, we decided to test clofoctol in K18-hACE2 transgenic mice. Female mice were inoculated intranasally (i.n.) with 5×10² TCID₅₀ of a clinical SARS-COV-2 isolate. The animals were then injected i.p. with clofoctol at 1 h and 8 h post-infection. This treatment was repeated twice the day after infection and some of the mice were sacrificed at day 2 post-inoculation (FIG. 1b, left panel). Remarkably, as compared to vehicle-treated animals, the infectious viral load detected in the lungs of clofoctol-treated mice was reduced by more than 1.1 log₁₀ at day 2 post-infection (FIG. 1b, middle panel). Analysis of viral RNA yields by RT-qPCR confirmed the reduced viral load in clofoctol-treated animals (FIG. 1b, right panel).

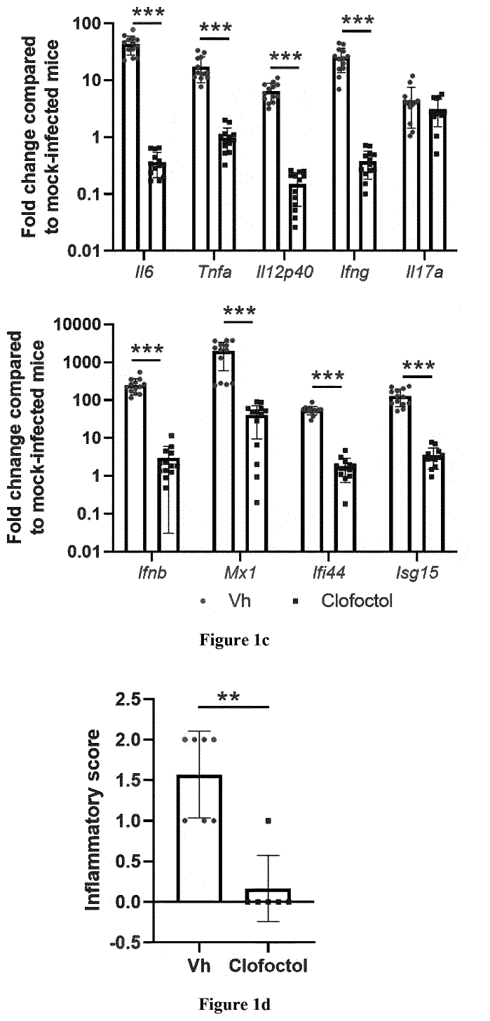

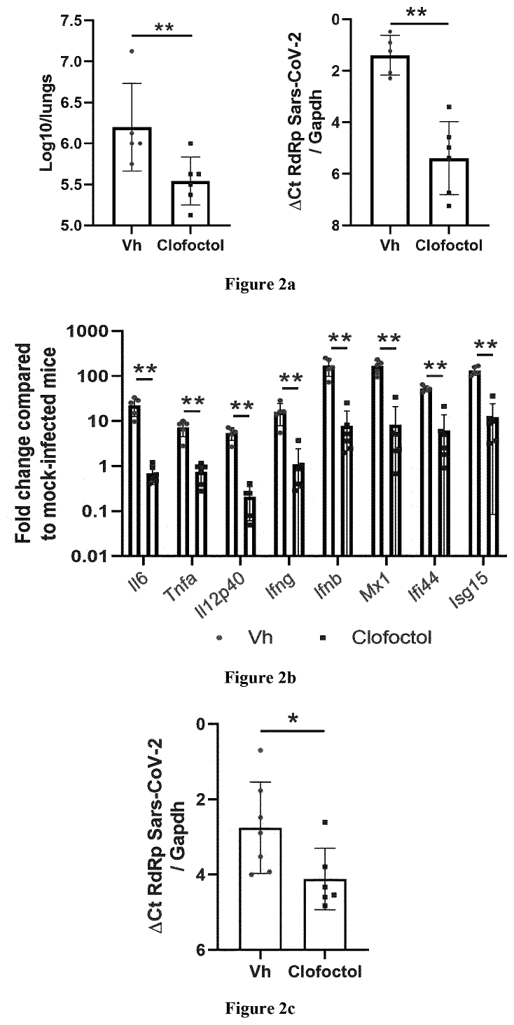

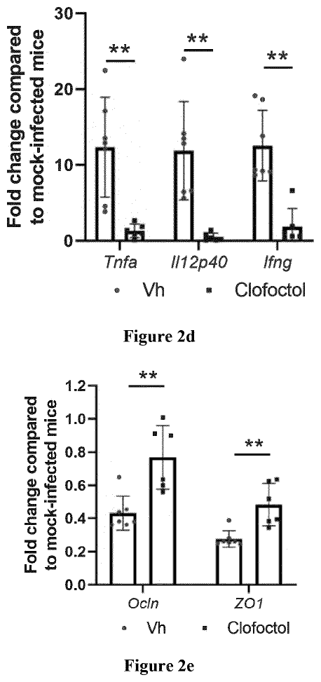

We then investigated whether the decrease in viral load would have positive effects on lung inflammation. Surprisingly, the expression of transcripts encoding IL-6, TNFα, IL12p40, IFNβ, IFNγ and the interferon-stimulated genes (ISG) Mx1, Ifi44 and ISG15 was markedly reduced in clofoctol-treated mice, in contrast with that of IL-17A (FIG. 1c). Similar results were also observed in male K18-hACE2 transgenic mice (FIGS. 2a and b). At day 4 post-inoculation, female mice treated during the first 2 days with clofoctol still showed a decrease in viral load (FIG. 2c). Expression of transcripts encoding inflammatory markers was also strongly decreased at day 4 post-infection (FIG. 2d. and data not shown). At this time point, SARS-COV-2 infection was associated with diminished expression of genes encoding markers of epithelial barrier function, including the tight-junction protein occludin (Ocln) and Zonula Occludens-1 (ZO1) (FIG. 2e). Interestingly, the drop of these transcript levels induced by SARS-COV-2 infection was significantly reduced in clofoctol-treated animals.

Lastly, we assessed the impact of clofoctol treatment on lung pathology at day 4 post-infection. In vehicle-treated animals, a mild multifocal broncho-interstitial pneumonia was observed (not shown). Signs of moderate inflammation, with the presence of neutrophils, macrophages and a few lymphocytes, were observed within alveolar lumens, inter-alveolar septa, and perivascular spaces which was accompanied by minimal perivascular edema. Slight vascular congestion and discrete intra-alveolar hemorrhages were also detected (not shown). In stark contrast, only a minimal interstitial inflammation was observed in clofoctol-treated mice (not shown), with a limited presence of macrophages and lymphocytes within inter-alveolar septa and little vascular congestion. Accordingly, clofoctol reduces the inflammatory score (FIG. 1d). We conclude that, at doses that produce lung concentrations close to those observed in human patients treated at the approved dose, clofoctol treatment in mice just after infection lowers SARS-COV-2 replication and, surprisingly, strongly reduces lung inflammation and pathological features associated with this viral infection.

### Statistical Analysis

Results are expressed as the mean #standard deviation (SD) unless otherwise stated. All statistical analysis was performed using GraphPad Prism software. A Mann-Whitney U test was used to compare two groups unless otherwise stated. Comparisons of more than two groups with each other were analyzed with the two-way ANOVA corrected for Dunnett's multiple comparisons test (viability test) or one-way ANOVA corrected for Dunnett's multiple comparisons test. p<0.05; **, p<0.01; ***, p<0.001. Sample sizes were dictated to adhere to the French home office 3R principles, while providing appropriate statistical power.

### EXAMPLE 2

### Clofoctol Lowers Inflammation Induced by LPS

To substantiate the surprisingly observation in another model, the inventors investigated the effect of clofoctol in several models of inflammation induced by lipopolysaccharide (LPS). To this end, MPI cells and THP1 cells were used in vitro and C57BL/6 mice were inoculated by the intra-nasal route with LPS.

### Cell Culture, Assessment of Gene Expression by Quantitative RT-PCR and ELISA

Max Plank Institute (MPI) cells are self-renewing and non-transformed cells originated from fetal liver of C57BL/6J mouse. MPI cells were used as a model of alveolar macrophages, due to their closer profile (Fejer et al., 2013). Cells were cultivated in RPMI Glutamax with 10% FBS, 1% Penicillin/streptomycin, and 30 ng/mL GM-CSF and incubated on 37° ° C.5% CO₂. MPI cells were used from passage 6 until passage 30 and they were negative for mycoplasma contamination, which was assessed with MycoAlert™ Mycoplasma Detection Kit (Lonza-Basel, Switzerland). For all experiments 1.5×10⁶ cells/mL were plated in the absence of GM-CSF and incubated for 3 h or overnight for adhesion. Various doses of clofoctol were added 30 minutes prior to stimulation with LPS (10 ng/ml, Escherichia coli O111:B4, Sigma). Cytokine production was measured from the supernatant of cells after 24 h of stimulation, accordingly to protocol's manufactures for IL-1β, IL-12p40, TNF-α, and MCP-1 (R&D Systems—Minneapolis, MN).

THP-1 designates a spontaneously immortalized monocyte-like cell line, derived from the peripheral blood of a childhood case of acute monocytic leukemia. Cells were cultivated in RPMI with HEPES without Glutamine complemented with 2 mM Glutamine, 10% FBS and 1% Penicillin/streptomycin and incubated at 37° C.-5% CO2. THP1 cells were used from passage 7 until passage 14 and they were negative for mycoplasma contamination, which was assessed with MycoAlert™ Mycoplasma Detection Kit (Lonza-Basel, Switzerland). For differentiation to a macrophage phenotype, THP-1 cells (1.106 cells/mL) were incubated with PMA diluted in medium (Phorbol 12-myristate 13-acetate) at 100 ng/mL for 72 h in 6-well plates (2 mL/well) for mRNA analysis or in 12 well-plates (750 μL/well) for ELISA analysis. Then Clofoctol and Dexamethasone were added at various concentrations (0.2% DMSO) 1 hour prior to lipopolysaccharide (LPS, Invitrogen™eBioscience™) stimulation at 100 ng/mL for 5 hours for mRNA analysis or for 24 hours for ELISA analysis in the culture medium with 0.2% FBS. In THP1, The levels of human cytokines were measured after 24 h of stimulation in the collected supernatants with human CCL2/MCP-1, IL-6 and TNF-α Duoset ELISA (R&D systems, Minneapolis, MN) according to the manufacturer's protocols.

Total RNA was extracted with NucleoSpin® RNA kit (Macherey-Nagel, Hoerdt, Germany). RNA (1 μg) was reverse-transcribed with the high-capacity cDNA reverse transcription kit (Applied Biosystems™, USA). The resulting cDNA was amplified using SYBR Green-based real time PCR Takyon™ No Rox SYBR® MasterMix dTTP Blue (Eurogentec™, Belgium) with the Light Cycler 480 (Roche). Relative quantification of MCP1, TNF-α, IL-6 and IL-1β was performed using the gene coding glyceraldehyde 3-phosphate dehydrogenase (GAPDH). Relative mRNA levels (2-ΔΔCt) were determined by comparing (a) the PCR cycle thresholds (Ct) for the gene of interest and the house keeping gene GAPDH (ΔCt) and (b) ΔCt values for treated and control groups (ΔΔCt). Data are expressed as a fold-increase over the mean gene expression level in solvent control wells.

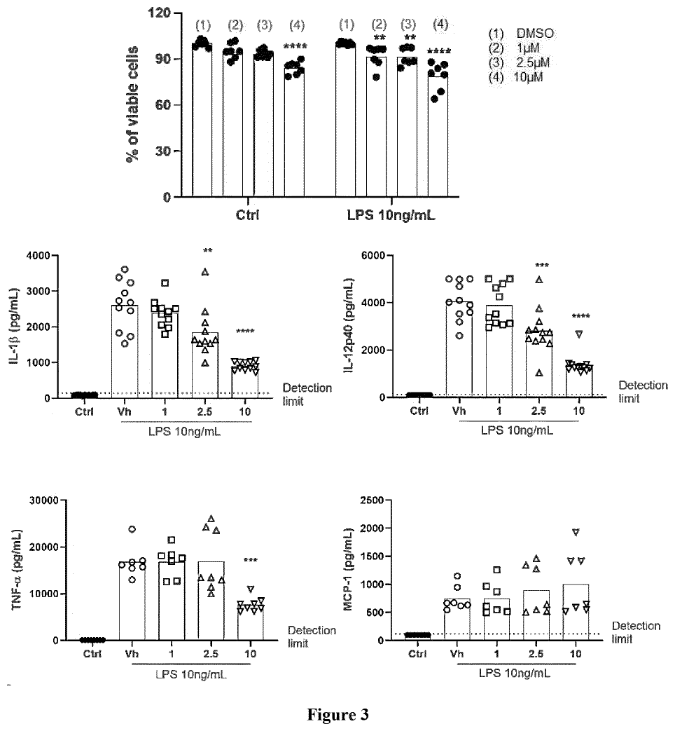

Clofoctol treatment reduces LPS-induced inflammation in macrophages. Alveolar macrophages are important sources of cytokine production in lungs. To assess whether clofoctol interferes with LPS-induced cytokine production by macrophages, the alveolar cell line MPI was used. Strikingly, clofoctol inhibited, in a dose dependent manner, the production of various inflammatory cytokines by macrophages, including IL-1β, IL-12p40 and TNF-α (FIG. 3). (Various doses of clofoctol were added 30 minutes prior to stimulation with LPS (10 ng/ml, Escherichia coli O111:B4, Sigma)).

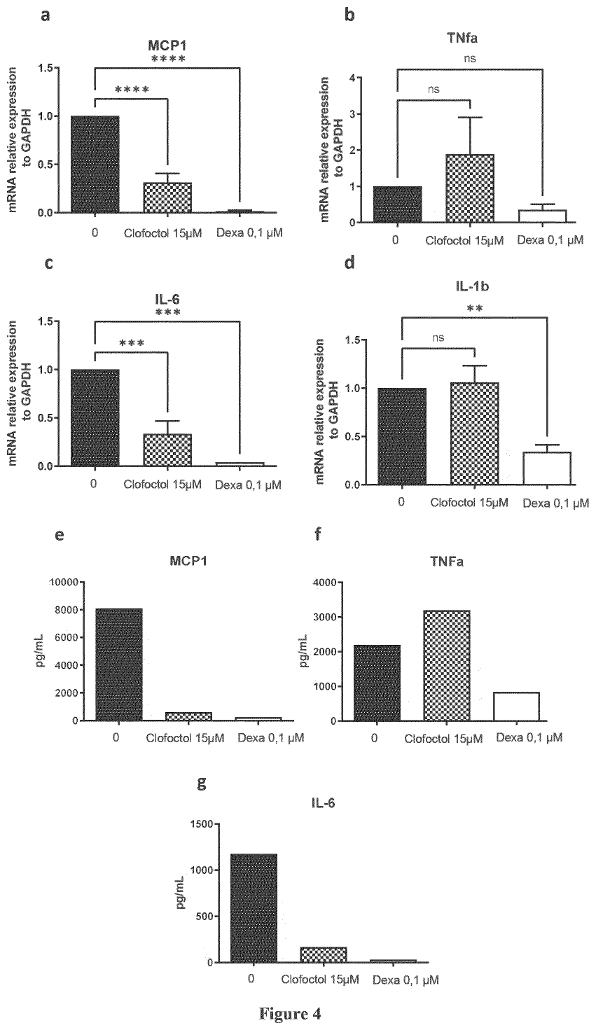

Clofoctol treatment reduces pro-inflammatory cytokines expression in LPS-induced macrophages differentiated from THP1 cells. We assessed whether clofoctol interferes with LPS induced cytokine production by PMA-differentiated THP-1 macrophages. Clofoctol is significantly reducing the expression of MCP1 and IL-6 genes (FIGS. 4a and c) and their corresponding protein (FIGS. 4e and g). At the time point used in the experiment, clofoctol is not significantly modulating IL1-β nor TNF-α (FIGS. 4b, 4d and 4f).

### Clofoctol Treatment in Mice Reduces LPS-Induced Inflammation

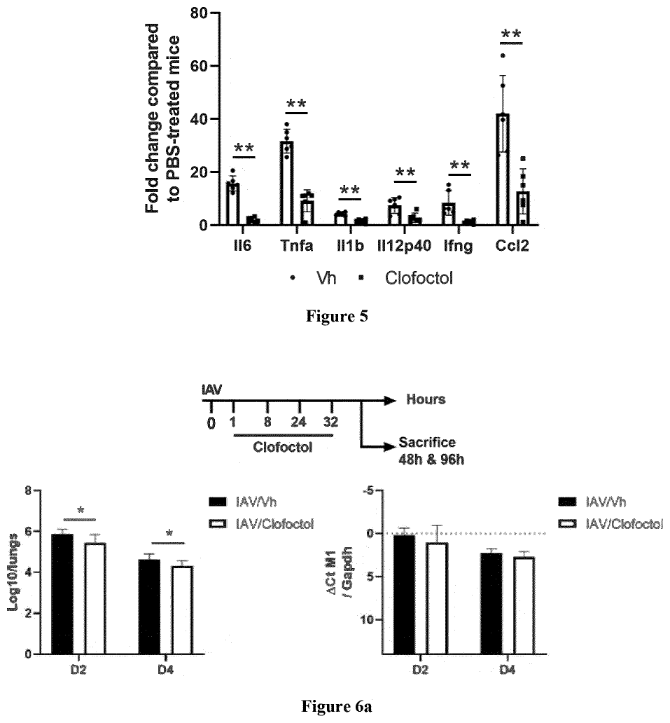

C57BL/6 mice were inoculated by the intra-nasal route with LPS (Escherichia coli 0111:B4) at 10 μg/mouse. Clofoctol was injected i.p. 1 h before and 8 h after LPS administration. Mice were sacrificed 24 hrs after LPS inoculation. The expression of transcripts encoding IL-6, TNFα, IL-1β, IFN-γ, IL12p40, and CCL2 was markedly reduced in clofoctol-treated mice (FIG. 5).

Therefore, clofoctol also inhibits inflammation in a non-infectious model.

### Statistical Analysis

Results are expressed as the mean #standard deviation (SD) unless otherwise stated. All statistical analysis was performed using GraphPad Prism software. A Mann-Whitney U test was used to compare two groups unless otherwise stated. Comparisons of more than two groups with each other were analyzed with the two-way ANOVA corrected for Dunnett's multiple comparisons test (viability test) or one-way ANOVA corrected for Dunnett's multiple comparisons test. p<0.05; **, p<0.01; ***, p<0.001. Sample sizes were dictated to adhere to the French home office 3R principles, while providing appropriate statistical power.

### EXAMPLE 3

### Mice and Ethics Statement

Specific pathogen-free C57BL/6J mice (6-8 week-old, male) were purchased from Janvier (Le Genest-St-Isle, France). Mice were maintained in a biosafety level 2 facility in the Animal Resource Center at the Lille Pasteur Institute for at least two weeks prior to usage to allow appropriate acclimation. All experiments complied with current national and institutional regulations and ethical guidelines (Institut Pasteur de Lille/B59-350009 and CEEA 75. Nord Pas-de-Calais). All experiments were approved by the Ministère de l'Education Nationale, de l'Enseignement Supérieur et de la Recherche, France (00357.03 and APAFIS 13743-2018022211144403).

### Infections and Clofoctol Treatment

For infection with IAV, mice were anesthetized by intramuscular injection of 1.25 mg of ketamine plus 0.25 mg of xylazine in 100 μl of phosphate buffered saline (PBS), and then intranasally (i.n.) infected with 50 μl of PBS containing (or not, in a mock sample) 100 p.f.u. of H1N1 A/California/04/2009 (pdm09) (Barthelemy et al., 2017, 2018). This dose corresponds a to sub-lethal dose. Clofoctol (50 mg/kg) was injected i.p. at 1 h and 8 h post-infection. The treatment was repeated the day after infection. Body weight was measured until day 2 post-infection. Mice were sacrificed at day 2 or day 4 post-infection. Survival and body weight were monitored daily after IAV infection and mice were euthanized when they lost in excess of 20% of their initial body weight. Regarding the long-term effect of clofoctol treatment, mice were treated once a day (50 mg/kg) during 4 days and sacrificed at day 28.

### Quantification of Viral Loads and Assessment of Gene Expression by Quantitative RT-PCR

Total RNA from lung tissues were extracted with the NucleoSpin® RNA kit (Macherey-Nagel, Hoerdt, Germany). RNA was reverse-transcribed with the High-Capacity cDNA Archive Kit (Life Technologies, USA). The resulting cDNA was amplified using SYBR Green-based real-time PCR and the QuantStudio™ 12K Flex Real-Time PCR Systems (Applied Biosystems™, USA) following manufacturers protocol. Relative quantification was performed using the gene coding glyceraldehyde 3-phosphate dehydrogenase (Gapdh). Specific primers were designed using Primer Express software (Applied Biosystems, Villebon sur Yvette, France). Relative mRNA levels (2^{-ΔΔCt}) were determined by comparing (a) the PCR cycle thresholds (Ct) for the gene of interest and the house keeping gene Gadph (ΔCt) and (b) ΔCt values for treated and control groups (ΔΔCt). Data are expressed as a fold-increase over the mean gene expression level in mock-treated mice. Quantification of viral RNA was performed as described in (Paget et al., 2011). Viral load is expressed as viral RNA normalized to gapdh expression level. Data were normalized against expression of the gapdh gene and were expressed as Ct.

### Statistical Analysis

Results are expressed as the mean±standard deviation (SD) unless otherwise stated. All statistical analysis was performed using GraphPad Prism software. A Mann-Whitney U test was used to compare two groups unless otherwise stated. Comparisons of more than two groups with each other were analyzed with the two-way ANOVA corrected for Dunnett's multiple comparisons test (viability test) or one-way ANOVA corrected for Dunnett's multiple comparisons test. p<0.05; **, p<0.01; ***, p<0.001. Sample sizes were dictated to adhere to the French home office 3R principles, while providing appropriate statistical power.

### Clofoctol Treatment in Mice Reduces Influenza-Associated Pulmonary Inflammation

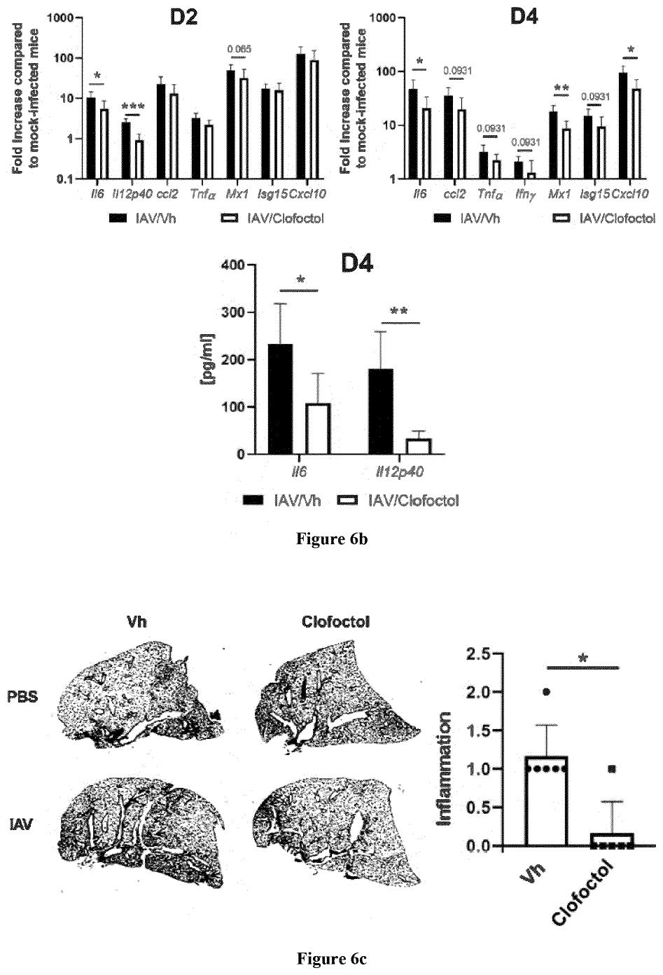

C57BL6/J male mice were intranasally (i.n.) infected with 50 μl of PBS containing (or not, in a mock sample) 100 p.f.u. of HIN1 A/California/04/2009 (pdm09) (Barthelemy et al., 2017, 2018). The animals were then injected intraperitoneally with clofoctol (50 mg/kg) at 1 h and 8 h post-infection. This treatment was repeated the day after infection. Mice were sacrificed at day 2 or day 4 post-infection (FIG. 6a, upper panel). As compared to untreated animals, the infectious viral load detected in the lungs of clofoctol-treated mice was slightly reduced at day 2 and day 4 post-infection (FIG. 6a, lower left panel). Analysis of viral RNA yields by RT-qPCR indicated a trend in reduced viral load in clofoctol-treated animals. (FIG. 6a, lower right panel). Despite this moderate antiviral effect, clofoctol treatment has a strong effect on lung inflammation. Remarkably, the expression of transcripts encoding IL-6, IL-12p40, Ccl2, IFN-γ and, to a lower extent, TNFα, was reduced in clofoctol-treated mice (FIG. 6b, upper panel and not shown). Reduced IL-6 and IL-12p40 production in lungs from clofoctol-treated mice was confirmed by ELISA (FIG. 6b, lower panel). In parallel, clofoctol treatment reduced the transcript expression of the interferon-stimulated genes (ISG) Mx1, ISG15 and Cxcl10. Lastly, we assessed the impact of clofoctol treatment on lung pathology. In vehicle-treated animals, a mild multifocal broncho-interstitial pneumonia was observed (FIG. 6c, arrows). Signs of moderate inflammation, with the presence of neutrophils, macrophages and a few lymphocytes, were observed within alveolar lumens, inter-alveolar septa, and perivascular spaces which was accompanied by minimal perivascular edema. In stark contrast, only a minimal interstitial inflammation was observed in clofoctol-treated mice, with a limited presence of macrophages and lymphocytes within inter-alveolar septa and little vascular congestion (FIG. 6c, lower panel for the inflammation scoring). We conclude that clofoctol treatment in mice reduces lung inflammation during influenza infection.

### Clofoctol Treatment in Mice Reduces Influenza-Associated Pulmonary Fibrosis

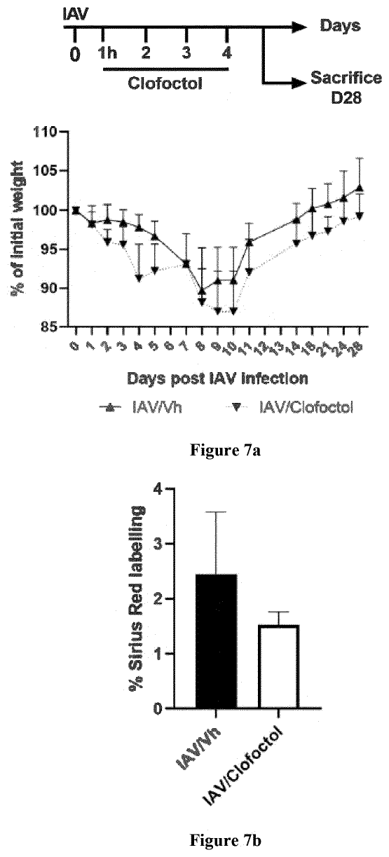

We then turned to analyze the long-term effect of clofoctol. To this end, mice were injected intraperitoneally with clofoctol (50 mg/kg) at 1 h post-infection and during the three following days (FIG. 7a, upper panel) (one injection per day). This protocol reduces body weight although in a much lower extent relative to the protocol used in FIG. 5 (FIG. 7a, lower panel and not shown). 28 days post-infection, mice were sacrificed and lung fibrosis was analyzed by Red Sirius staining. Interestingly, mice treated with clofoctol tended to develop less fibrosis relative to controls (FIG. 7b).

### CONCLUSION

In conclusion, we herein demonstrate that clofoctol treatment drastically reduced inflammation in several relevant models. The data show that the drastic anti-inflammatory effects of the molecule are independent from the anti-viral effects and thus justify to suggest that the molecule would be suitable for the treatment of inflammation in general. The data also demonstrate that clofoctol prevents inflammation-induced fibrosis.

### REFERENCES

Throughout this application, various references describe the state of the art to which this invention pertains. The disclosures of these references are hereby incorporated by reference into the present disclosure.

1. A method of treating inflammation in a subject in need thereof comprising administering to the subject a therapeutically effective amount of clofoctol.

2. The method of claim 1 wherein the inflammation is localized in the gastrointestinal tract, kidneys, liver, heart, skin, spleen, brain, kidney and/or pulmonary tract.

3. The method of claim 1 wherein the subject suffers from an inflammatory bowel disease.

4. The method of claim 1 wherein the subject suffers from a skin inflammation.

5. The method of claim 1 wherein the subject suffers from a lung inflammation.

6. The method of claim 5 wherein the subject suffers from a cystic fibrosis, asthma or COPD.

7. The method of claim 1 wherein the subject suffers from myocarditis.

8. The method of claim 1 wherein the inflammation is secondary to infection caused by virus, bacteria, fungi, and/or parasites.

9. The method of claim 8 wherein the inflammation is secondary to a viral infection.

10. The method of claim 9 wherein the inflammation is not secondary to a SARS-COV2 infection.

11. The method of claim 1 wherein the subject suffers from sepsis.

12. The method of claim 5 wherein the subject suffers from a lung inflammation caused by a lung infection.

13. The method of claim 12 wherein the lung inflammation infection is caused by a bacterium selected from the group consisting of Streptococcus pneumoniae, Staphylococcus aureus, Streptococcus agalactiae, Streptococcus pyogenes, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Burkholderia cepacia, Burkholderia pseudomallei, Bacillus anthracis, Bacillus cereus, Bordetella pertussis, Stenotrophomonas maltophilia, a bacterium from the citrobacter family, a bacterium from the ecinetobacter family, and Mycobacterium tuberculosis and Mycobacterium abscessus.

14. The method of claim 12 wherein the lung inflammation infection is caused by a virus selected from the group consisting of influenza virus, respiratory syncytial virus, adenovirus, metapneumovirus, cytomegalovirus, parainfluenza virus, and smallpox.

15. The method of claim 12 wherein the subject suffers from an acute respiratory distress syndrome.

16. The method of claim 1 wherein the subject suffers from a multisystem inflammatory syndrome.

17. The method of claim 16 wherein the subject suffers from a multisystem inflammatory syndrome (MIS-C) with severe myocarditis.

18. A method of preventing inflammation-induced fibrosis in a subject in need thereof comprising administering to the subject a therapeutically effective amount of clofoctol.

19. The method of claim 3 wherein the inflammatory bowel disease is Crohn's disease, ulcerative colitis or irritable bowel syndrome.

20. The method of claim 14 wherein the influenza virus is Influenza virus A or Influenza virus B; the parainfluenza virus is hPIV-1, hPIV-2, hPIV-3 or hPIV-4; and the coronavirus is a SARS-coronavirus.

21. The method of claim 14 wherein the SARS-coronavirus is SARS-Cov1 or SARS-Cov2.
